(19) United States
(12) Patent Application Publication
(10) Pub. No.: US 2023/0284978 A1
(43) Pub. Date: Sep. 14, 2023

(54) Detection and Differentiation of Activity Using Behind-the-Ear Sensing

(71) Applicants: Earable, Inc., Boulder, CO (US); The Regents of the University of Colorado, a body corporate, Denver, CO (US)

(72) Inventors: Tam Vu, Boulder, CO (US); Galen Pogoncheff, San Anselmo, CA (US)

(21) Appl. No.: 18/163,278

(22) Filed: Feb. 1, 2023

Related U.S. Application Data
(60) Provisional application No. 63/305,359, filed on Feb. 1, 2022.

(51) Int. Cl. A61B 5/00; A61B 5/0205

(52) U.S. Cl. CPC A61B 5/6815; A61B 5/0205; A61B 5/7282; A61B 5/7267; A61B 5/332

(57) ABSTRACT

Novel tools and techniques are provided for the detection and differentiation of activities and/or conditions based on measured bio-signals.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates, in general, to methods, systems, and apparatuses for facial muscle and movement detection.

### BACKGROUND

Many conventional wearables integrate sensor technology to provide health information such as heart rate. This information can be continuously gathered by the wearable as the user goes through his or her normal daily activities. The ability to gather additional bio-signal information and process the gathered information presents several significant challenges in the art.

Typically, a sleep study or other forms of patient monitoring are utilized to diagnose and treat sleep disorders. Polysomnography (PSG) and camera-based solutions, for example, have been used to detect sleep disorders, using eye and facial movement in particular, measuring electrical signals from the human head, such as brain waves, eyeball movements, chin muscle tone, and behaviors such as eyelid closures, eye blinks, and head nods. Tests and studies are typically conducted by technicians in a clinical environment.

Therefore, methods, systems, and apparatuses for are provided for speech, chewing, and swallowing detection using wearable device sensing are provided.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 is a schematic diagram illustrating a system for detecting and differentiating activities, in accordance with various embodiments;

FIG. 2 is a flow diagram illustrating a method for detecting and differentiating activities, in accordance with various embodiments; and

FIG. 3 is a block diagram illustrating an exemplary computer or system hardware architecture, in accordance with various embodiments.

FIGS. 4A-4C illustrate components of a signal processing and feature extraction pipeline, in accordance with various embodiments.

FIGS. 5A-5C illustrate techniques for reducing dimensionality of figures and visualizing the features, in accordance with various embodiments.

FIG. 6A illustrates activity level classification scores, in accordance with some embodiments.

FIG. 6B illustrates feature attribution analysis, in accordance with some embodiments.

FIG. 7 illustrates activity-level classification architecture of a machine learning model, in accordance with various embodiments

### DETAILED DESCRIPTION OF CERTAIN EMBODIMENTS

Various embodiments provide tools and techniques for detecting and differentiating activity.

An outstanding problem in medicine continues to be a lack of physician-administered patient reported outcomes (PROs) that are currently used in clinic to assess patient muscle weakness symptoms such as difficulty of swallowing. Such qualitative assessments are often inadequate (i.e., they do not measure true patient symptoms) or inaccurate (i.e., the same test administered on the same subject produces different results). The inadequate aspects of these PROs are due to the qualitative assessment itself; the absence of quantitative tools highlight an unmet medical need to be able to measure patient muscle weakness more accurately and quantitatively. Current tools that exist to perform quantitative analysis of fascial muscle activity have significant limitations. Polysomnography (PSG) is an invasive and expensive procedure, and while effective as a diagnostic tool for sleep disorders, both convenience and cost necessitate the need for better alternative approaches. Furthermore, PSG and similar clinical solutions do not facilitate such measurements in a familiar environment, which may reduce diagnostic and evaluation accuracy.

Additional limitations of current qualitative assessments are that they are subject to the experience and bias of physicians. A major issue with PRO questionaries is the lack of adjustment for the time that a subject has had the disease. Subjects who have had neurological diseases for a decade may not indicate in a PRO that they have difficulty, for example, swallowing. Patients tend to respond in the negative if they have adapted to the disease. In such clinical scenario, the true symptoms fail to be captured.

Various embodiments disclosed herein provide systems, devices, apparatus, and methods that can measure certain symptoms quantitatively and relatively accurately (e.g., chewing, swallowing) may potentially serve as an excellent supportive tool to facilitate physicians assessing symptoms more accurately.

In an aspect, a system for detecting and differentiating one or more activities is provided. In another aspect, an apparatus for detecting and differentiating one or more activities is provided. In a further aspect, a method for detecting and differentiating one or more activities is provided.

For example, a system in accordance with some embodiments might comprise a processor and a computer readable medium in communication with the processor. The system might include one or more sensors. In some cases, a sensor might be situated behind and/or above the ear of a user. The sensor might be part of an apparatus, such as a wearable device, that can include the sensor and one or more other devices, such as another sensor situated behind the other ear of the user. In some cases, the apparatus might include one or more speakers and/or microphones. In an aspect, one or more of the speakers and/or microphones can include bone conduction elements to provide for emission or collection of audio and/or other frequencies through bone conduction. In fact, the sensor, in some cases, can include such a microphone and/or speaker.

In an aspect of some embodiments, the computer readable medium has encoded thereon a set of instructions executable by the processor to perform a set of operations. Such operations can include operations can include obtaining, via a sensor (e.g., one or more of sensors described above and elsewhere herein), a first signal from a first position of a user (which can include, without limitation, a patient, an owner of the sensor, and/or the like). In some cases, the operations can include separating the first signal into one or more component bio-signals. In some cases, the bio-signals can include one or more of an electroencephalogram (EEG) signal, electrooculography (EOG) signal, and/or electromyography (EMG) signal.

The operations might further include extracting one or more features from each of the one or more individual bio-signals, and/or determining, based on the one or more features extracted from the one or more individual bio-signals, whether the patient is engaged in one or more activities or is in a particular position.

In some cases, the set operations might include determining whether the patient is engaged in a first activity based on the determination that the patient is engaged in the one or more activities. For instance, determining whether the patient is engaged in the first activity further can include determining a first score for a first set of features associated with the first activity; the first score can indicate how closely the one or more features extracted from the one or more individual bio-signals matches the first set of features. Any number of activities can be detected, including without limitation speaking, chewing, and/or swallowing. In some embodiments, the operations can include applying a stimulus to the patient, e.g. in response to the determination that the patient is engaged in the first activity.

In some cases, the operations can include diagnosing whether the patient is afflicted with a first condition. This operation might comprise determining a first score for a first set of features associated with a first activity while the patient is engaged in the first activity. The first score can indicated how closely the one or more features extracted from the one or more individual bio-signals matches the first set of features. The operations can then include determining whether the first score meets a threshold score for the first activity; if it does not, the operations can include determining that the patient is afflicted with the first condition. A number of conditions can be detected this way, including without limitation neurodegenerative and/or neuromuscular disease, neurological injuries and/or trauma of the face, a dental condition, a nutritional condition, and/or a mental health condition.

As noted above, in some cases, the system includes an apparatus such as a wearable device, which can comprise the sensor; in an aspect, the wearable device and/or the sensor can be configured to be in contact with the skin of the user. For instance, the wearable device can be configured to position one or more sensors above the ear of the patient and below the crown of the patient. Additionally and/or alternatively, the wearable device is configured to position one or more sensors on the skin over the mastoid bone of the patient. In some embodiments, the wearable device is configured to be worn around an ear of the patient. In some embodiments, he wearable device is a headband. The apparatus might comprises the processor and/or the computer readable medium. In other cases, the apparatus might be configured to communication, via wired and/or wireless communications, with the processor.

In another set of embodiments, the set of operations might include obtaining a plurality of reference signals from a reference population. The plurality of reference signals might correspond to reference signals obtained from the reference population while engaged in speech, chewing, and swallowing; in other activities as disclosed herein. Alternatively and/or additionally, the plurality of reference signals might correspond to reference signals obtained from a population of subjects having one or more conditions, such as the conditions described herein. The operations might include separating each reference signal of the plurality of reference signals into a respective set of one or more component bio-signals and/or extract a respective feature set from each set of one or more component bio-signals. These respective feature sets can be used to train a machine learning model.

The system might employ a machine learning model comprising one or more computational blocks, such as one or more random forest classifiers, one or more convolutional neural network, and/or one or more transformer networks. Training the machine learning model might include associating the respective feature set with a respective ground truth, wherein the respective ground truth corresponds to activity and/or condition, such as those described herein. The operations can include generating, e.g., via the machine learning model, respective sets of features for each of the one or more component bio-signals, each of which can be associated with one or more activities and/or conditions, respectively;

The features extracted from the bio-signals obtained from the user (e.g., using the process(es) described above and/or elsewhere herein) then can be used to determine whether the user is engaged in a first activity of the one or more activities based, at least in part, on the one or more extracted features. For example, the one or more extracted features can be passed to the machine learning model, which can be configured to determine a respective similarity score of the one or more extracted features to each of the one or more sets of features including a first set of features associated with the first activity. The operations then can include differentiating the first activity from other activities of the one or more activities based, at least in part, on the respective similarity scores of the one or more extracted features.

Additionally and/or alternatively, the same technique (or similar techniques) can be used to different a condition of a user from one or more candidate conditions (including without limitation those described herein. In some embodiments, the operations can include determining a subset of component bio-signals comprising features indicative of the first activity and/or condition, wherein the subset of component bio-signals includes the one or more component bio-signals. As noted above, individual bio-signals can include, without limitation, an electroencephalogram (EEG) signal, electrooculography (EOG) signal, and/or electromyography (EMG) signal.

Thus, some embodiments provide systems that can be configured (e.g., with instructions on a computer readable medium) to perform any combination of the operations described above and/or elsewhere herein. Other embodiments can include non-transitory computer readable media having stored thereon computer software comprising a set of instructions that are executable by one or more processors to perform a set of operations, including any combination of the operations described above and/or elsewhere herein. Still other embodiments can include methods, including without limitation methods comprising any combination of the operations described above and/or elsewhere herein.

While various aspects and features of certain embodiments have been summarized above, the following detailed description illustrates a few exemplary embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments may be practiced without some of these specific details. In other instances, certain structures and devices are shown in block diagram form. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

Exemplary Methods

To the extent any abstract concepts are present in the various embodiments, those concepts can be implemented as described herein by devices, software, systems, and methods that involve specific novel functionality (e.g., steps or operations), such as detecting speech, chewing, and swallowing from signals collected by sensors of a wearable device, which may be worn by a patient at home, or outside of the clinical environment.

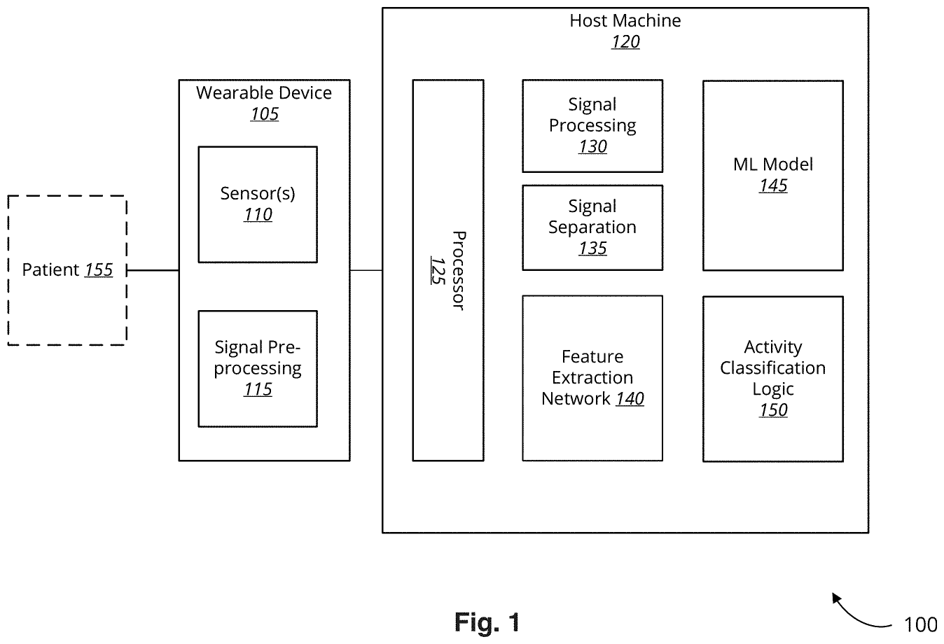

FIG. 1 illustrates a system 100 for detecting and differentiating activity, in accordance with various embodiments. The system 100 includes wearable device 105, one or more sensors 110, signal pre-processing logic 115, host machine 120, processor 125, signal processing logic 130, signal separation logic 135, feature extraction network 140, machine learning (ML) model 145, and activity classification logic 150. It should be noted that the various components of the system 100 are schematically illustrated in FIG. 1, and that modifications to the system 100 may be possible in accordance with various embodiments.

In various embodiments, the wearable device 105 may be coupled to a user, (e.g., a patient, etc.) 155. Thus, the wearable device 105 may be configured to be worn by the patient 155. The one or more sensors 110 may be configured to make contact with the skin of the patient 155 when worn. Accordingly, in some embodiments, the wearable device 105 may include, without limitation, one or more ear pieces, a headband, mask, goggles, glasses, cap, hat, visor, or helmet.

Thus, the one or more sensors 110 may be configured to be coupled to various parts of the body of the patient 155. As previously described, in various embodiments, the one or more sensors 110 may be configured to be coupled to skin behind-the-ears of the patient 155, above the ears and below the crown of the head of the patient. In further embodiments, the one or more sensors 110 may be configure d to be coupled to the skin of the patient on the mastoid bone of the patient 155. In further embodiments, the one or more sensors 110 may further be configured to other parts of the patient, including, without limitation, the eyes, eyelids, and surrounding areas around the eyes, forehead, temple, the mouth and areas around the mouth, chin, scalp, and neck of the patient 155. In some embodiments, the one or more sensors 110 may further include adhesive material to attach the one or more sensors 110 to the skin of the patient 155.

In various embodiments, the wearable device 105 may comprise one or more sensors 110, which may be configured to collect bio-signals from the patient 155. In various embodiments, the one or more sensors 110 may be positioned within the ear pieces so as to make contact with the skin of the patient 155. For example, in some embodiments, the one or more sensors 110 may be configured to make contact with the skin of the patient 155 behind the earlobe of the patient 155, above the ears of the patient 155, or other locations around the ear of the patient 155. In some examples, this may include at least one sensor of the one or more sensors 110 being in contact with the skin covering the respective mastoid bones of the patient 155.

In various embodiments, the one or more sensors 110 may include various types of sensors, including, without limitation, contact electrodes and other electrode sensors (such as, without limitation, silver fabric, copper pad, or gold-plated copper pad), optical sensors and photodetectors (including a light source for measurement), microphones and other sound detectors (e.g., a bone-conductive microphone, a MEMS microphone, an electret microphone), water sensors, pH sensors, salinity sensors, skin conductance sensors, heart rate monitors, pulse oximeters, and other physiological signal sensors. In yet further embodiments, the one or more sensors 120 may further include one or more positional sensors and/or motion sensors, such as, without limitation, an accelerometer, gyroscope, inertial measurement unit (IMU), global navigation satellite system (GNSS) receiver, or other suitable sensor.

Accordingly, in various embodiments, the one or more sensors 110 may be configured to detect one or more bio-signals, including, without limitation, brain waves (EEG), eyes movements (EOG), facial muscle activities (EMG), from areas above and behind human ears. Further bio-signals may include, without limitation, electro-dermal activity (EDA), heart rate, respiratory rate, sounds from inner body such as speech, breathing sounds, and organ's sounds. In some embodiments, the one or more sensors 110 may be configured to capture the various bio-signals respectively through different types of sensors. In some embodiments, the one or more sensors 110 may be configured to measure the one or more bio-signals from behind-the-ear.

The signal collected by the one or more sensors 110 may be a combined or composite signal comprising one or more component bio-signals. For example, a signal collected by the one or more sensors 110 may be a composite signal comprising EEG, EOG, and EMG signals. Accordingly, once the measured composite signal is provided to the host machine 120, signal separation logic 135 may be configured to separate the composite signal into one or more component bio-signals. In some examples, the signal separation logic 135 may be configured to separate an EEG, EOG, and EMG signal from the composite bio-signal.

The feature extraction network 140 may be configured to extract features from each of the component bio-signals, such as EEG, EOG, and EMG. Accordingly, in some embodiments, the feature extraction network 140 may extract a respective feature set corresponding to each of EEG, EOG, EMG, or other component bio-signals, respectively. The feature set generated by the feature extraction network 140 may be fed to an ML model 145 for further processing.

In various embodiments, the ML model 145 may include, without limitation, a decision tree based learning model, such as a random forest classifier. In further embodiments, an artificial neural network, such as a feed forward network, convolutional neural network (CNN) including recurrent neural networks, transformer networks, or other suitable neural networks.

As described in further detail in the Appendix, according to various embodiments, The ML model 145 may be trained based on reference data collected from a reference population. Reference data may be used to collect bio-signals from the reference population and extract relevant features from selected bio-signals (e.g., EEG, EOG, and EMG). In some embodiments, the ML model 145 may be trained to classify activities, such as speech, chewing, and swallowing, by using a random forest classifier. Further examples of activities, and conditions are provided below and in the Appendix. The activities, such as speech, chewing, and swallowing, may be detected and differentiated from each other, and other activities, based on the identified features of the selected bio-signals.

Thus, the component bio-signals obtained from the patient may be fed to the ML model 145, which may then generate a score representing similarity to the feature sets associated with each respective activity, such as speech, chewing, and swallowing. Accordingly, in various embodiments, the ML model 145 may determine, based on the respective scores, whether the patient 155 is engaged in one or more activities, such as speaking, chewing, or swallowing. In some embodiments, the ML model 145 may be configured to provide, as an output, the respective scores for each activity, such as speech, chewing, and swallowing, to the activity classification logic 150. The activity classification logic 150 may, in some examples, be configured to determine whether the patient 155 is engaged in one or more activities, such as speaking, chewing, or swallowing, based on one or more algorithms (e.g., weighting, normalization, or other processing of data from the ML model 145). In yet further embodiments, the ML model 145 and/or activity classification logic 150 may be configured to determine the disorders and/or abnormal patterns of speech, chewing, and swallowing. For example, in some examples, the ML model 145 and/or activity classification logic 150 may be able to detect that various levels of swallowing occurs during speech or chewing, or that speech occurs during swallowing or chewing, or that chewing occurs during speech or swallowing, when such activities should not be occurring concurrently.

In yet further embodiments, the ML model 145 may be configured to provide scores related to other conditions. Conditions may include, without limitation, neurodegenerative and neuromuscular diseases, such as myasthenia gravis, Grave's disease, multiple sclerosis, and Parkinson's disease; neurological injury and/or trauma to the face, such as Bell's palsy, stroke, and shingles; dental issues, such as temporomandibular joint dysfunction (TMJ syndrome); nutritional conditions, such as obesity, diabetes, poor nutrition, malnutrition, eating disorders, dehydration; and mental health conditions, such as anxiety and depression; and other conditions such as dysfunctional swallowing and pneumonia.

In some examples, diagnosing a mental health condition may be based on inference of facial expressions demonstrating emotions associated with an emotional state of the patient and/or mental health condition, and scoring produced by the ML model 145 for the related facial expressions. Accordingly, in some examples, scores related to facial expressions associated with an emotional state of the patient may be used by the activity classification logic 150 to diagnose a mental health condition. In some examples, the activity classification logic 150 may further be configured to diagnose the various conditions above based on scores for various activities (or deficits in the various activities). In yet further examples, the activity classification logic 150 may be configured to quantify a response to and/or adherence by the patient to a therapy/treatment regime for the various conditions. This may include tracking of historic activity (e.g., historic scores for various activities) and/or changes in the performance of the activity over time (e.g., a change in historic score over time).

In yet further embodiments, the activity classification logic 150 may further be configured to track and monitor therapeutic patient outcome measures and responses based on the detected one or more activities. For example, signals of speech, chewing, swallowing, inferences of facial expressions, associations with emotional states, eye gaze and eye movements, and other activities may be used to develop a patient outcome assessment. In some examples, the one or more detected activities may further be combined with changes in medication, activities, or other physiological states to determine a patient outcome measure. In some examples, the patient outcome measures may be determined individually, based on individual-specific detected activities, historic activity, patient history, and individual-specific changes in activities, medications, etc. In other embodiments, the patient outcome measure for a patient may be determined based on collective data determined from a population. For example, a sample population may exhibit changes in activity and/or physiological states associated with an improvement or worsening of a patient outcome measure. Accordingly, in various embodiments, patient outcome measures may be determine algorithmically, as described above, and monitored intermittently and/or continuously in clinical and in at-home settings.

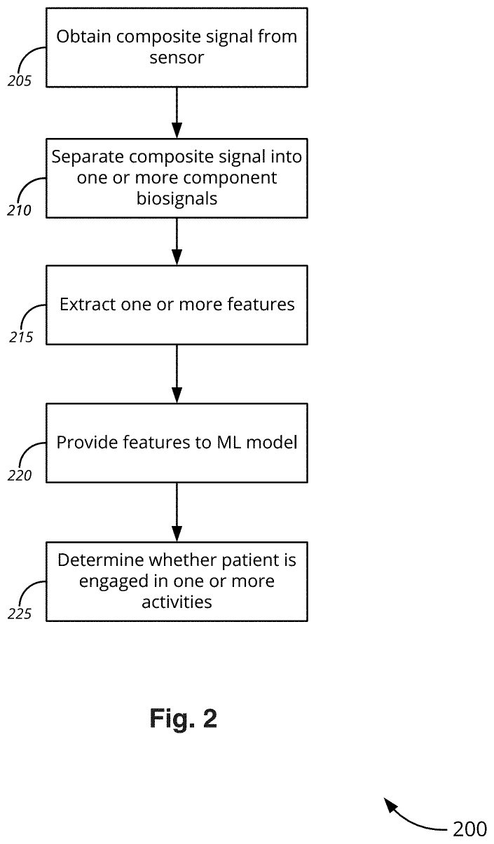

FIG. 2 is a flow diagram illustrating a method 200 for detecting and differentiating activity. The method 200 may include, at block 205, by obtaining a composite signal from a sensor (e.g., a sensor of one or more sensors). The method 200 continues, at block 210, by separating the composite signal into one or more component bio-signals. As previously described, the component bio-signals may include EEG, EOG, and/or EMG signals. At block 215, the method 200 continues by extracting one or more features for each of the component bio-signals. In some examples, a set of one or more extracted features may be generated for each component bio-signal, respectively. Thus, one or more features may be extracted from each bio-signal respectively. At block 220, the one or more extracted features may be fed to the ML model to determine respective scores for each of one or more activities, such as speech, chewing, and swallowing. As described in greater detail in the Appendix, the ML model may be trained to identify and classify activities based on features identified from reference bio-signals. In some embodiments, the ML model may be trained using a random forest classifier model. In some examples, the ML model may be a neural network, such as a feed forward network (FFN), CNN including recurrent neural networks (RNN), or a transformer network. The method 200 continues, at block 225, by determining whether the patient is engaged in one or more activities, based on the score generated by the ML model.

Exemplary Computer Systems

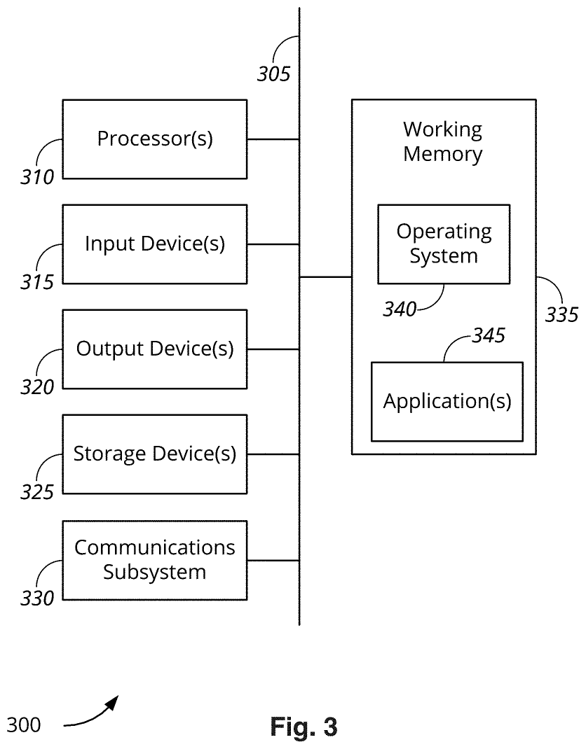

FIG. 3 is a block diagram illustrating an exemplary computer or system hardware architecture, in accordance with various embodiments. FIG. 3 provides a schematic illustration of one embodiment of a computer system 300 that can perform the methods provided by various other embodiments, as described herein, and/or can perform the functions of computer or hardware system, as described above. It should be noted that FIG. 3 is meant only to provide a generalized illustration of various components, of which one or more (or none) of each may be utilized as appropriate. FIG. 3, therefore, broadly illustrates how individual system elements may be implemented in a relatively separated or relatively more integrated manner.

The computer or hardware system 300—which may represent an embodiment of the computer or hardware system described above with respect to FIG. 1—is shown comprising hardware elements that can be electrically coupled via a bus 305 (or may otherwise be in communication, as appropriate). The hardware elements may include one or more processors 310, including, without limitation, one or more general-purpose processors and/or one or more special-purpose processors (such as microprocessors, digital signal processing chips, graphics acceleration processors, and/or the like); one or more input devices 315, which can include, without limitation, a mouse, a keyboard, and/or the like; and one or more output devices 320, which can include, without limitation, a display device, a printer, and/or the like.

The computer or hardware system 300 may further include (and/or be in communication with) one or more storage devices 325, which can comprise, without limitation, local and/or network accessible storage, and/or can include, without limitation, a disk drive, a drive array, an optical storage device, solid-state storage device such as a random access memory (“RAM”) and/or a read-only memory (“ROM”), which can be programmable, flash-updateable, and/or the like. Such storage devices may be configured to implement any appropriate data stores, including, without limitation, various file systems, database structures, and/or the like.

The computer or hardware system 300 may also include a communications subsystem 330, which can include, without limitation, a modem, a network card (wireless or wired), an infra-red communication device, a wireless communication device and/or chipset (such as a Bluetooth™ device, an 802.11 device, a WiFi device, a WiMax device, a WWAN device, cellular communication facilities, etc.), and/or the like. The communications subsystem 330 may permit data to be exchanged with a network (such as the network described below, to name one example), with other computer or hardware systems, and/or with any other devices described herein. In many embodiments, the computer or hardware system 300 will further comprise a working memory 335, which can include a RAM or ROM device, as described above.

The computer or hardware system 300 also may comprise software elements, shown as being currently located within the working memory 335, including an operating system 340, device drivers, executable libraries, and/or other code, such as one or more application programs 345, which may comprise computer programs provided by various embodiments (including, without limitation, hypervisors, VMs, and the like), and/or may be designed to implement methods, and/or configure systems, provided by other embodiments, as described herein. Merely by way of example, one or more procedures described with respect to the method(s) discussed above may be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code may be encoded and/or stored on a non-transitory computer readable storage medium, such as the storage device(s) 325 described above. In some cases, the storage medium may be incorporated within a computer system, such as the system 300. In other embodiments, the storage medium may be separate from a computer system (i.e., a removable medium, such as a compact disc, etc.), and/or provided in an installation package, such that the storage medium can be used to program, configure, and/or adapt a general purpose computer with the instructions/code stored thereon. These instructions may take the form of executable code, which is executable by the computer or hardware system 300 and/or may take the form of source and/or installable code, which, upon compilation and/or installation on the computer or hardware system 300 (e.g., using any of a variety of generally available compilers, installation programs, compression/decompression utilities, etc.) then takes the form of executable code.

Exemplary Device Configuration

In one embodiment, the sensor(s) (which can be part of a wearable device) acquire EEG, EMG, and EOG bio-signals via 4 dry electrodes, located at scalp locations directly above the left and right ears and on left and right mastoid processes. This electrode placement yields raw bio-signal data analogous to that which could be acquired at EEG reference locations T3, T4, M1, and M2 of the 10-20 electrode placement positions). These locations enable high fidelity acquisition of EMG activity from activation of the Temporalis and surrounding muscle groups, EEG activity from electrical activity in the brain, and EOG signals yielded by eye deflections. Meanwhile, this electrode configuration is comfortable and nonintrusive to support everyday use. Six virtual channels (2 EEG, 2 EMG, and 2 EOG) are derived from these 4 electrodes following signal referencing, scaling, and filtering. These 6 channels enable analysis of brain, eye, and muscle activation, respectively. In order to acquire detailed EEG, EMG, and EOG signals, this bio-signal data is sampled at a rate of 250 Hz.

Exemplary Processing Pipeline

An exemplary processing pipeline might proceed as follows: Following signal re-referencing, scaling, filtering, and separation, the signals of each virtual channel are segmented based on the presence/absence of facial movement activity. Statistical measures, DSP analysis, and handcrafted feature computations are then preformed on the resulting signal components. Most general features were processed to summarize each waveform, with the exception of a subset of features that are specific to EMG, EOG, or EEG activity.

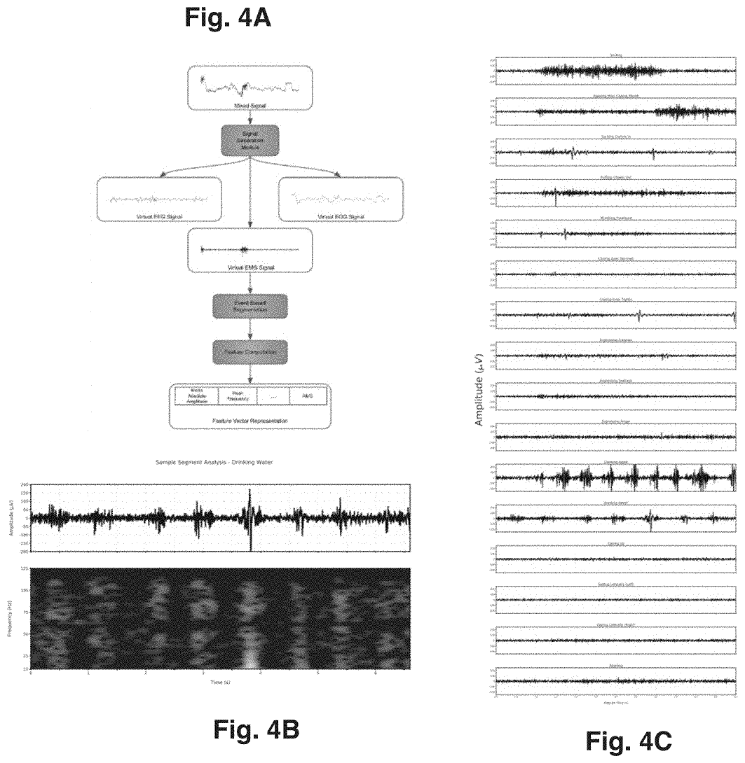

For example, by reference to FIG. 4, a signal separation module is applied to the mixed signal derived from the wearable device, to decompose the EEG, EMG, and EOG waves into their component parts (FIG. 4A). These signals are then subject to an event-based segmentation algorithm, and features are extracted. (FIG. 4B). Time and frequency representations of EMG activity resulting from a subject drinking water. This plot shows around 6.5 s of EMG data in both the time (top) and frequency (bottom) domains. (FIG. 4C). EMG activity visualized in the time domain over 16 activities.

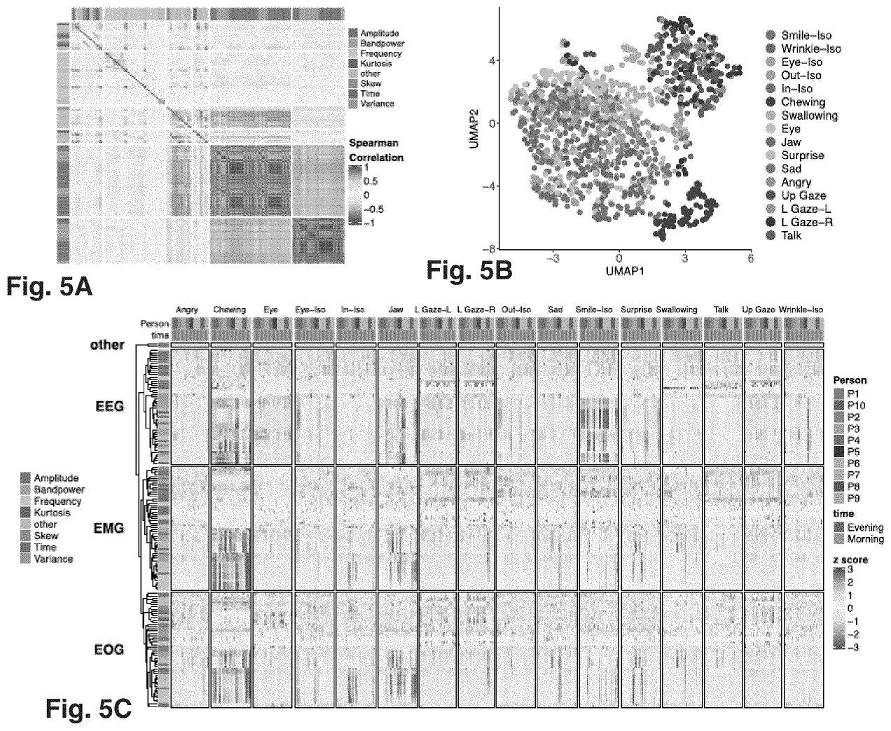

In some aspects, wearable devices in accordance with various embodiments produces continuous waveform data, and features are processed from these waves. FIG. 5 illustrates the isolation of parameters that measure aspects of eye and facial movement. Some embodiments analyze the relationship between all of the parameters across all 16 PerfO activities by performing Spearman correlations of all parameters against each other. To determine the optimal number of parameter groups, one can perform K means clustering of the Spearman correlations and determine 6 unique clusters of parameters (FIG. 5A—Spearman correlation of all features against each other, represented as a heatmap. K=6 clusters from K means clustering (optimal number) are shown. All 16 activities were pooled for this analysis.). Amplitude and Bandpower parameters tended to cluster together into two of the six clusters, while other parameters like those from the frequency domain clustered separately. These clusters indicate that the parameters may be measuring different components of the signal in the PerfO activities. UMAP dimensionality reduction was performed to evaluate qualitative differences between the 16 PerfO activities, across each subject and timepoint, using all 161 bio-signal parameters, demonstrating differences between the 16 activities (FIG. 5B—UMAP dimension reduction of all 161 features. Each individual trial repeat is a point on this graph. The color of the point represents the activities performed during that trial.). While there is overlap between some of the activities, activities like swallowing clearly separate out from the rest. Heatmaps can demonstrate differences between the activities for different classes of parameters (FIG. 2C—Heatmap of all 161 features (rows) for all trial repeats (columns). Columns are sorted first by the 16 activities, and within each activity, by subject, and then time of day when the activity was performed.). Taken together, these results demonstrate the utility of the various embodiments to generate parameters that may describe, and allow for measurement of, unique PerfO activities.

Exemplary Machine Learning Model

To develop a model in accordance with some embodiments, a number of trials are performed on test subjects. In some embodiments, To guarantee reliable ground truth data annotations, data from each trial is manually labeled by an expert technician. For each trial, the onset and offset endpoints of each performed activity are annotated accordingly. A time-synchronized video recording of the subject is utilized as a reference source in this annotation procedure. Using these activity annotations, signals are then segmented according to noted onset and offset timestamps. Statistical measures, DSP analysis, and handcrafted feature computations, are finally preformed on the resulting signal segments.

Spearman correlations between all parameters and all activities were computed. The silhouette method can be used to determine the optimal number of clusters with the factoextra package in R with function fviz_nbclust with 100 bootstrapped samples. Spearman correlation results are reported for all parameters pooled. For each of the 16 activities, for all of the 161 bio-signal parameters, the number of walks analyzed (n), the minimum value (min), maximum value (max), median value (median), mean value (mean), standard deviation of the mean (sd), and standard error of the mean (se).’

Dimensionality reduction of the parameters can be performed in Python with umap-learn, with an effective minimum distance between embedded points of one, and default parameters. UMAP coordinates were plotted in ggplot2 in R. Heatmaps of parameters are displayed with individual trials as columns and parameters as rows. All heatmaps display Z scored parameters in the rows, computed across all activities (see FIG. 2C). All clustering on heatmaps is supervised and ordered by subject and time of day.

To investigate how the measured data could be used to classify each of the 16 activities, or each of the subjects, one can employ multi-class classification models using the Python sklearn module. In some embodiments, model selection in the context of 10-fold cross validation a random forest classifier (using the sklearn RandomForestClassifier class) was chosen, with 500 decision trees. In each classification setting (activity- and subject-level classification), model training and validation can be performed using a 80% of the dataset while the remaining 20% of the dataset with withheld for testing. Data samples can be assigned to one of the two subsets at random to reduced bias in evaluation results. The Random Forest classification model can be constructed to detect each activity from the other 15 activities (1-against-all). Activity detection F1 scores can be used as the primary metric for evaluating model performance.

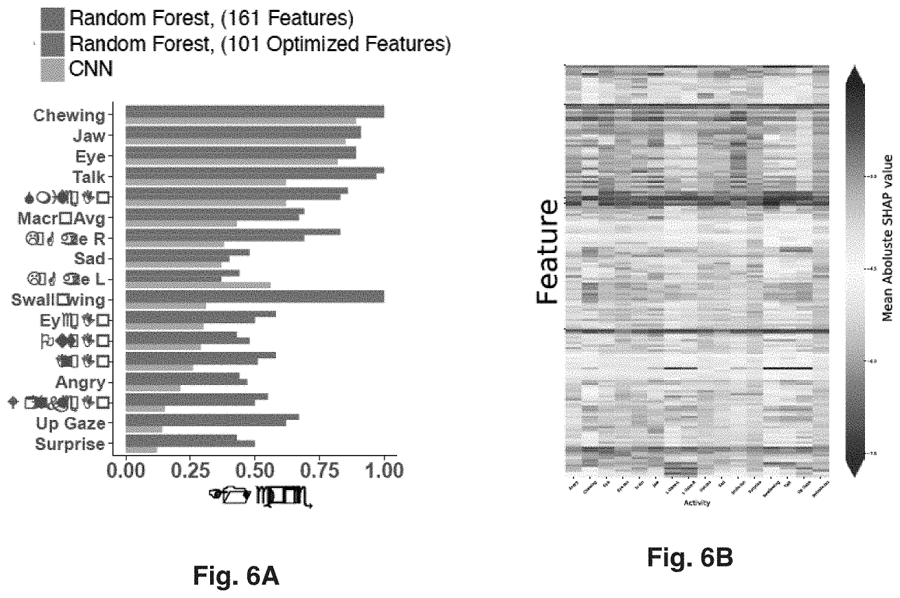

Following developmental evaluation on training validation datasets, two primary models can be built for activity-level classification. The first can use all 161 measured bio-signal features in the model. The second can use an optimized set of reference features, with feature reduction performed with the Bourta package. Briefly, all 161 measured bio-signal features are copied, called shadow features, and their labels are shuffled. Each shadow feature is compared to itself for 1,000 iterations of classification, and only features that perform better than chance are kept. This analysis indicated an optimized set of 101 features that may be used for classification. Finally, to evaluate how well these features perform relative to using low level representations of the waveform data, CNN models can be built with the data to classify the 16 PerfO activities (FIG. 6A—Activity level classification F1 scores for all bio-signal parameters (161 features), Boruta selected features (101 features), and using raw waveform data (CNN)). In this modeling, fixed size spectrograms (which quantify how the power at given frequencies changes as a function of time) computed from the PerfO signal segments can be used as model input.

To explain the underlying features that are most important for the RF models, feature attribution analysis with Shapley additive explanations (SHAP) can be used. For a specific activity prediction, the SHAP value of a feature is computed as the change in the expected value of the model output when this feature is observed, compared to when it is missing for the test set predictions. The effect of each feature as it is added to the model is summed and averaged across all 161 parameters used. The parameters are represented as the Mean average SHAP value and are shown in a heatmap (as a log 10). (FIG. 6B—Feature attribution analysis using SHAP (SHapley Additive exPlanations) values for each feature (row) for each activity (columns)).

In recent years, deep learning models have been used to achieve high performance in many tasks relevant to classification of bio-signal data. Among the many popular deep learning architectures leveraged in such tasks, convolutional neural networks (CNNs) are widely used for their ability to learn patterns in structured, multidimensional data (e.g., time-frequency signal representations). In applying such methodologies to the task of PerfO activity-level classification, 16-class CNN classification models can be constructed developed and analyzed. These CNN models might be constructed to map 2-dimensional spectrogram representations of the PerfO activity signal segments to a probability distribution over the 16 classes.

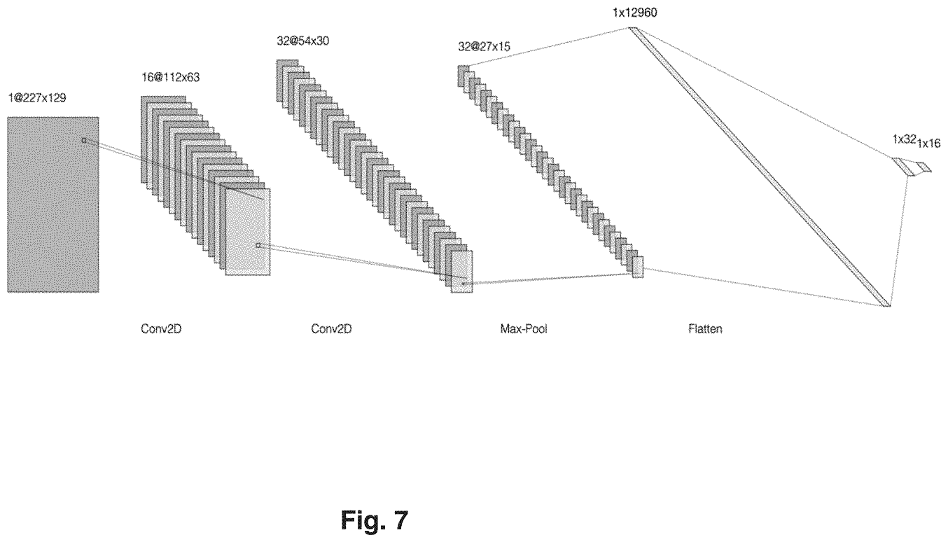

In some cases, because deep learning models often require large datasets to learn generalizable functions, data augmentation can maximize the diversity in the training set. For instance, each time a signal segment is read into the training data set, multiple random croppings of this segment also can be added to the training set. To an extent, this allows one to increase the size of the training dataset without collecting additional samples, helping to counter overfitting. To maintain constant length input signals among the PerfO activities that varied in duration, activity segments shorter in duration than the fixed input data duration (30 seconds) can be repeated after shifting the segment according to the randomized cropping scheme, while segments longer in duration can be truncated to the fixed input data duration via randomized cropping. Data augmentation generally should not performed for the testing set, however, as it would bias the resulting model performance estimate. Additional techniques applied to reduce model variance can include the use of L2 kernel regularization in the convolutional and fully connected model layers and the inclusion of dropout layers throughout the network. FIG. 7 illustrates the activity-level classification architecture of the model in accordance with some embodiments.

### CONCLUSION

It will be apparent to those skilled in the art that substantial variations may be made in accordance with specific requirements. For example, customized hardware (such as programmable logic controllers, field-programmable gate arrays, application-specific integrated circuits, and/or the like) may also be used, and/or particular elements may be implemented in hardware, software (including portable software, such as applets, etc.), or both. Further, connection to other computing devices such as network input/output devices may be employed.

As mentioned above, in one aspect, some embodiments may employ a computer or hardware system (such as the computer or hardware system 300) to perform methods in accordance with various embodiments of the invention. According to a set of embodiments, some or all of the procedures of such methods are performed by the computer or hardware system 300 in response to processor 310 executing one or more sequences of one or more instructions (which may be incorporated into the operating system 340 and/or other code, such as an application program 345) contained in the working memory 335. Such instructions may be read into the working memory 335 from another computer readable medium, such as one or more of the storage device(s) 325. Merely by way of example, execution of the sequences of instructions contained in the working memory 335 may cause the processor(s) 310 to perform one or more procedures of the methods described herein.

The terms “machine readable medium” and “computer readable medium,” as used herein, refer to any medium that participates in providing data that causes a machine to operate in a specific fashion. In an embodiment implemented using the computer or hardware system 300, various computer readable media may be involved in providing instructions/code to processor(s) 310 for execution and/or may be used to store and/or carry such instructions/code (e.g., as signals). In many implementations, a computer readable medium is a non-transitory, physical, and/or tangible storage medium. In some embodiments, a computer readable medium may take many forms, including, but not limited to, non-volatile media, volatile media, or the like. Non-volatile media includes, for example, optical and/or magnetic disks, such as the storage device(s) 325. Volatile media includes, without limitation, dynamic memory, such as the working memory 335. In some alternative embodiments, a computer readable medium may take the form of transmission media, which includes, without limitation, coaxial cables, copper wire, and fiber optics, including the wires that comprise the bus 305, as well as the various components of the communication subsystem 330 (and/or the media by which the communications subsystem 330 provides communication with other devices). In an alternative set of embodiments, transmission media can also take the form of waves (including without limitation radio, acoustic, and/or light waves, such as those generated during radio-wave and infra-red data communications).

Common forms of physical and/or tangible computer readable media include, for example, a floppy disk, a flexible disk, a hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read instructions and/or code.

Various forms of computer readable media may be involved in carrying one or more sequences of one or more instructions to the processor(s) 310 for execution. Merely by way of example, the instructions may initially be carried on a magnetic disk and/or optical disc of a remote computer. A remote computer may load the instructions into its dynamic memory and send the instructions as signals over a transmission medium to be received and/or executed by the computer or hardware system 300. These signals, which may be in the form of electromagnetic signals, acoustic signals, optical signals, and/or the like, are all examples of carrier waves on which instructions can be encoded, in accordance with various embodiments of the invention.

The communications subsystem 330 (and/or components thereof) generally will receive the signals, and the bus 305 then may carry the signals (and/or the data, instructions, etc. carried by the signals) to the working memory 335, from which the processor(s) 305 retrieves and executes the instructions. The instructions received by the working memory 335 may optionally be stored on a storage device 325 either before or after execution by the processor(s) 310.

While certain features and aspects have been described with respect to exemplary embodiments, one skilled in the art will recognize that numerous modifications are possible. For example, the methods and processes described herein may be implemented using hardware components, software components, and/or any combination thereof. Further, while various methods and processes described herein may be described with respect to particular structural and/or functional components for ease of description, methods provided by various embodiments are not limited to any particular structural and/or functional architecture but instead can be implemented on any suitable hardware, firmware and/or software configuration. Similarly, while certain functionality is ascribed to certain system components, unless the context dictates otherwise, this functionality can be distributed among various other system components in accordance with the several embodiments.

Moreover, while the procedures of the methods and processes described herein are described in a particular order for ease of description, unless the context dictates otherwise, various procedures may be reordered, added, and/or omitted in accordance with various embodiments. Moreover, the procedures described with respect to one method or process may be incorporated within other described methods or processes; likewise, system components described according to a particular structural architecture and/or with respect to one system may be organized in alternative structural architectures and/or incorporated within other described systems. Hence, while various embodiments are described with—or without—certain features for ease of description and to illustrate exemplary aspects of those embodiments, the various components and/or features described herein with respect to a particular embodiment can be substituted, added and/or subtracted from among other described embodiments, unless the context dictates otherwise. Consequently, although several exemplary embodiments are described above, it will be appreciated that the invention is intended to cover all modifications and equivalents within the scope of the following claims.

What is claimed is:

1. A system comprising:
a processor; and
a computer readable medium in communication with the processor, the computer readable medium having encoded thereon a set of instructions executable by the processor to:
obtain, via a sensor, a first signal from a first position of a patient;
separate the first signal into one or more component bio-signals;
extract one or more features from each of the one or more individual bio-signals; and
determine, based on the one or more features extracted from the one or more individual bio-signals, whether the patient is engaged in one or more activities.

2. The system of claim 1, wherein the one or more individual bio-signals includes at least one of an electroencephalogram (EEG) signal, electrooculography (EOG) signal, electromyography (EMG) signal.

3. The system of claim 1, wherein the set of instructions is further executable by the processor to:
determine whether the patient is engaged in a first activity based on the determination that the patient is engaged in the one or more activities, wherein determining whether the patient is engaged in the first activity further comprises determining a first score for a first set of features associated with the first activity, wherein the first score indicates how closely the one or more features extracted from the one or more individual bio-signals matches the first set of features.

4. The system of claim 3, wherein the set of instructions is further executable by the processor to:
apply a stimulus to the patient in response to the determination that the patient is engaged in the first activity.

5. The system of claim 3, wherein the first activity is one of speaking, chewing, or swallowing.

6. The system of claim 1, wherein the set of instructions is further executable by the processor to:
diagnose whether the patient is afflicted with a first condition, wherein diagnosing whether the patient is afflicted with the first condition further comprises:
determining a first score for a first set of features associated with a first activity while the patient is engaged in the first activity, wherein the first score indicates how closely the one or more features extracted from the one or more individual bio-signals matches the first set of features;
determining whether the first score meets a threshold score for the first activity; and
wherein if the threshold score is not met, determining that the patient is afflicted with the first condition.

7. The system of claim 6, wherein the first condition comprises a neurodegenerative or neuromuscular disease.

8. The system of claim 6, wherein the first condition comprises a neurological injury or trauma of the face.

9. The system of claim 6, wherein the first condition comprises a dental condition.

10. The system of claim 6, wherein the first condition comprises a nutritional condition.

11. The system of claim 6, wherein the first condition comprises a mental health condition.

12. The system of claim 1, further comprising a wearable device, the wearable device comprising the sensor, the sensor configured to be in contact with the skin of the patient.

13. The system of claim 12, wherein the wearable device is configured to position the sensor above the ear of the patient and below the crown of the patient, wherein the first position is a position located above the ear of the patient and below the crown of the patient.

14. The system of claim 12, wherein the wearable device is configured to position the sensor on the skin over mastoid bone of the patient, wherein the first position is a position over the mastoid bone of the patient.

15. The system of claim 12, wherein the wearable device is configured to be worn around an ear of the patient.

16. The system of claim 12, wherein the wearable device is a headband.

17. The system of claim 1, wherein:
the one or more extracted features are passed to the machine learning model, wherein the machine learning model is configured to determine a respective similarity score of the one or more extracted features to each of the one or more sets of features including a first set of features associated with the first activity; and
the set of instructions is further executable by the processor to:
obtain a plurality of reference signals from a reference population, the plurality of reference signals corresponding to reference signals obtained from the reference population while engaged in speech, chewing, and swallowing;
separate each reference signal of the plurality of reference signals into a respective set of one or more component bio-signals;
extract a respective feature set from each set of one or more component bio-signals;
train a machine learning model with the respective feature set, wherein training the machine learning model includes associating the respective feature set with a respective ground truth, wherein the respective ground truth corresponds to speech, chewing, or swallowing; and
differentiate the first activity from other activities of the one or more activities based, at least in part, on the respective similarity scores of the one or more extracted features.

18. The system of claim 17, wherein the set of instructions is further executable by the processor to:
determine a subset of component bio-signals comprising features indicative of the first activity, wherein the subset of component bio-signals includes the one or more component bio-signals.

19. The system of claim 17, wherein the machine learning model is a random forest classifier.

21. The system of claim 17, wherein the machine learning model is a convolutional neural network.

22. The system of claim 17, wherein the machine learning model is a transformer network.

23. A non-transitory computer readable medium having stored thereon computer software comprising a set of instructions that, when executable by a processor to:
obtain, via a sensor, a first signal from a first position of a patient;
separate the first signal into one or more component bio-signals;
extract one or more features from each of the one or more individual bio-signals; and
determine, based on the one or more features extracted from the one or more individual bio-signals, whether the patient is engaged in one or more activities.

24. A method comprising:
obtaining, via a sensor, a first signal from a first position of a patient;
separating the first signal into one or more component bio-signals;
extracting one or more features from each of the one or more individual bio-signals; and
determining, based on the one or more features extracted from the one or more individual bio-signals, whether the patient is engaged in one or more activities.
